(19) United States
(12) Patent Application Publication
(10) Pub. No.: US 2013/0039852 A1
(43) Pub. Date: Feb. 14, 2013

(54) METHODS OF TARGETING CELLS FOR DIAGNOSIS AND THERAPY

(71) Applicant: NORTHEASTERN UNIVERSITY, Boston, MA (US)

(72) Inventor: Ban-An KHAW, Milton, MA (US)

(73) Assignee: NORTHEASTERN UNIVERSITY, Boston, MA (US)

(21) Appl. No.: 13/644,862

(22) Filed: Oct. 4, 2012

Related U.S. Application Data
(62) Division of application No. 12/467,845, filed on May 18, 2009, now abandoned.
(60) Provisional application No. 61/053,733, filed on May 16, 2008.

(51) Int. Cl. A61K 39/395; G01N 33/53; A61K 51/10; G01N 33/574; A61P 35/00; A61K 49/00; C12N 5/071

(52) U.S. Cl. 424/1.49; 424/9.1; 435/7.21; 424/178.1; 435/375; 435/7.23

(57) ABSTRACT

Methods of making bispecific binding complexes and nanopolymers coupled to detection and/or therapeutic agents are disclosed. Also disclosed are methods of using such bispecific binding complexes and nanopolymers for detecting and treating cells.

### CROSS-REFERENCE TO RELATED APPLICATIONS

This application is a divisional application of U.S. application Ser. No. 12/467,845, filed May 18, 2009, which claims the benefit of U.S. Provisional Application No. 61/053,733, filed May 16, 2008, the contents of both of which are hereby incorporated herein in their entirety.

### BACKGROUND OF THE INVENTION

Small cancerous lesions are difficult to detect in vivo due to the high background activity, the low target activity, as well as the limited specificity of the targeting agents. The high background activity is due to the ionic interaction of drug molecules with the oppositely charged surfaces of cells and cellular matrices in vivo. The low target activity is due to the limitation of the number of signal moieties that can be loaded on individual drug molecules.

Current standard cancer therapies include surgery, chemotherapy, radiation, and autologous cell transplantation. Surgery may generally effective in the early treatment of cancer. However, metastatic growth of tumors can prevent any complete cure. Chemotherapy, which involves administration of compounds having antitumor activity, while effective in the treatment of some cancers, is often accompanied by severe side effects, including nausea and vomiting, bone marrow depression, renal damage, and central nervous system depression. Radiation therapy has also been used to target cancer cells, as cancer cells are less able to repair themselves after treatment with radiation. However, radiation cannot be used to treat many cancers because of the sensitivity of normal cells which surround cancerous tissue.

Thus, what is needed are improved methods for the diagnosis and treatment of cancer.

### SUMMARY OF THE INVENTION

The invention is based, at least in part, on the discovery that a bispecific binding complex can be used to target a nanopolymer to a target cell. Accordingly, in one aspect, the invention features a method of detecting a cancer cell in a subject, the method comprising: (a) administering to the subject a bispecific binding complex; (b) administering to the subject a polymer comprising an detection agent, the bispecific binding complex specifically binding the polymer and the bispecific binding complex specifically binding an antigen on the cancer cell; and (c) detecting the detection agent, thereby detecting the cancer cell.

In some embodiments, the bispecific binding complex comprises a first antibody covalently linked to a second antibody, the first antibody specifically binding the polymer and the second antibody specifically binding the antigen on the cancer cell. In particular embodiments, the first antibody and the second antibody are linked by a thioether bond, a disulfide bond, a peptide bond, or an ester bond.

In some embodiments, the polymer is coupled to an antigen. In certain embodiments, the antigen coupled to the polymer is diethylene triaminepentaacetic acid (DTPA), ethylene diamine tetraacetic acid (EDTA), dinitrophenol, or 1,4,7,10-tetraazacyclododecane-N,N′,N″,N′″-tetraacetic acid (DOTA). In some embodiments, the first antibody specifically binds the antigen coupled to the polymer.

In some embodiments, the polymer is polylysine, polyglutamic acid, or N-(2-hydroxypropyl)methacrylamide.

In certain embodiments, the detection agent is a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium) technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

In some embodiments, the cancer cell is a squamous cancer cell cancer, lung cancer cell, peritoneum cancer cell, hepatocellular cancer cell, gastrointestinal cancer cell, pancreatic cancer cell, glioblastoma cell, cervical cancer cell, ovarian cancer cell, liver cancer cell, bladder cancer cell, hepatoma cell, breast cancer cell, colon cancer cell, rectal cancer cell, colorectal cancer cell, endometrial cancer cell, uterine carcinoma cell, salivary gland carcinoma cell, kidney or renal cancer cell, prostate cancer cell, vulval cancer cell, thyroid cancer cell, hepatic carcinoma cell, anal carcinoma cell, or penile carcinoma cell.

In some embodiments, the cancer cell antigen is a pan cancer antigen, HER-2 receptor antigen, EGF receptor antigen, VEGF receptor antigen, or gastrin releasing peptide receptor antigen.

In certain embodiments, the subject is a human, ape, monkey, orangutan, chimpanzee, dog, cat, guinea pig, rabbit, rat, mouse, horse, cattle, or cow.

In another aspect, the invention features a method of delivering a chemotherapeutic agent to a cancer cell, the method comprising: (a) contacting the cancer cell with a bispecific binding complex; and (b) contacting the cancer cell with a polymer coupled to the chemotherapeutic agent, the bispecific binding complex specifically binding the polymer, and the bispecific binding complex specifically binding an antigen on the cancer cell, the chemotherapeutic agent thereby being delivered to the cancer cell.

In some embodiments, the bispecific binding complex comprises a first antibody covalently linked to a second antibody, the first antibody specifically binding the polymer, and the second antibody specifically binding the antigen on the cancer cell. In particular embodiments, the first antibody and the second antibody are linked by a thioether bond, a disulfide bond, a peptide bond, or an ester bond.

In some embodiments, the polymer is coupled to an antigen. In certain embodiments, the antigen coupled to the polymer is diethylene triaminepentaacetic acid (DTPA), ethylene diamine tetraacetic acid (EDTA), dinitrophenol, or 1,4,7,10-tetraazacyclododecane-N,N′,N″,N′″-tetraacetic acid (DOTA). In some embodiments, the first antibody specifically binds the antigen coupled to the polymer.

In some embodiments, the polymer is polylysine, polyglutamic acid, or N-(2-hydroxypropyl)methacrylamide.

In certain embodiments, the chemotherapeutic agent is 6 mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, doxorubicin, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, l-dehydrotestosterone, glucocorticoids, procaine, tetracaine, lidocaine, propranolol, puromycin, or calicheamicin.

In some embodiments, the cancer cell is a squamous cancer cell cancer, lung cancer cell, peritoneum cancer cell, hepatocellular cancer cell, gastrointestinal cancer cell, pancreatic cancer cell, glioblastoma cell, cervical cancer cell, ovarian cancer cell, liver cancer cell, bladder cancer cell, hepatoma cell, breast cancer cell, colon cancer cell, rectal cancer cell, colorectal cancer cell, endometrial cancer cell, uterine carcinoma cell, salivary gland carcinoma cell, kidney or renal cancer cell, prostate cancer cell, vulval cancer cell, thyroid cancer cell, hepatic carcinoma cell, anal carcinoma cell, or penile carcinoma cell.

In some embodiments, the cancer cell antigen is a pan cancer antigen, HER-2 receptor antigen, EGF receptor antigen, VEGF receptor antigen, or gastrin releasing peptide receptor antigen.

In some embodiments, the cancer cell is in a subject, and the chemotherapeutic agent is administered to the subject. In certain embodiments, the subject is a human, ape, monkey, orangutan, chimpanzee, dog, cat, guinea pig, rabbit, rat, mouse, horse, cattle, or cow. In other embodiments, the chemotherapeutic agent is delivered to the cell in vitro.

In other embodiments, the polymer further comprises a detection agent, and the method further comprises detecting the detection agent, thereby detecting the cancer cell. In certain embodiments, the detection agent is a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In) technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

In yet another aspect, the invention features a method of detecting a cell, the method comprising: (a) contacting the cell with an antibody covalently linked to a ligand; (b) contacting the cell with a polymer comprising an detection agent, the antibody specifically binding the polymer, and the ligand specifically binding a receptor on the cell; and (c) detecting the detection agent, thereby detecting the cell.

In particular embodiments, the antibody and the ligand are linked by a thioether bond, a disulfide bond, a peptide bond, or an ester bond.

In some embodiments, the polymer is coupled to an antigen. In certain embodiments, the antigen coupled to the polymer is diethylene triaminepentaacetic acid (DTPA), ethylene diamine tetraacetic acid (EDTA), dinitrophenol, or 1,4,7,10-tetraazacyclododecane-N,N′,N″,N′″-tetraacetic acid (DOTA). In some embodiments, the antibody specifically binds the antigen coupled to the polymer.

In some embodiments, the polymer is polylysine, polyglutamic acid, or N-(2-hydroxypropyl)methacrylamide.

In some embodiments, the ligand is bombesin.

In some embodiments, the detection agent is a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

In certain embodiments, contacting steps (a) and (b) comprise administering the antibody and the polymer to a subject. In certain embodiments, the subject is a human, ape, monkey, orangutan, chimpanzee, dog, cat, guinea pig, rabbit, rat, mouse, horse, cattle, or cow. In other embodiments, the cell is detected in vitro.

In yet another aspect, the invention features a method of treating a cell, the method comprising: (a) contacting the cell with an antibody covalently to a ligand, the ligand specifically binding a receptor on the cell; and (b) contacting the ligand-bound cell with a polymer coupled to a therapeutic agent, the antibody specifically binding the polymer, the therapeutic agent thereby being delivered to, and treating, the cell.

In particular embodiments, the antibody and the ligand are linked by a thioether bond, a disulfide bond, a peptide bond, or an ester bond.

In some embodiments, the polymer is coupled to an antigen. In certain embodiments, the antigen coupled to the polymer is diethylene triaminepentaacetic acid (DTPA), ethylene diamine tetraacetic acid (EDTA), dinitrophenol, or 1,4,7,10-tetraazacyclododecane-N,N′,N″,N′″-tetraacetic acid (DOTA). In some embodiments, the antibody specifically binds the antigen coupled to the polymer.

In some embodiments, the polymer is polylysine, polyglutamic acid, or N-(2-hydroxypropyl)methacrylamide.

In some embodiments, the ligand is bombesin.

In other embodiments, the cell is in a subject, and the antibody-ligand conjugate and the polymer-therapeutic agent complex are administered to the subject.

In certain embodiments, the subject is a human, ape, monkey, orangutan, chimpanzee, dog, cat, guinea pig, rabbit, rat, mouse, horse, cattle, or cow. In other embodiments, the cell is treated in vitro.

In certain embodiments, the chemotherapeutic agent is 6 mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, doxorubicin, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, l-dehydrotestosterone, glucocorticoids, procaine, tetracaine, lidocaine, propranolol, puromycin, or calicheamicin.

In certain embodiments, the polymer further comprises a detection agent, and the method further comprises detecting the detection agent, thereby detecting the cell. In some embodiments, the detection agent is a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

### BRIEF DESCRIPTION OF THE DRAWINGS

The foregoing and other objects of the present invention, the various features thereof, as well as the invention itself, may be more fully understood from the following description, when read together with the accompanying drawings, in which:

FIG. 1 is a schematic representation of targeting using a bispecific antibody complex and radiolabeled negatively charged polymers.

FIG. 2 is a graphic representation of the elution profiles of bispecific antibodies, monospecific antibodies, and F(ab′)₂ by Ultrogel-AcA22 column chromatography.

FIG. 3 is a graphic representation of the elution profiles of Doxorubicin covalently linked to polyglutamic acid nanopolymers (Dox-DPG) and free Doxorubicin using Sephadex G-25 column chromatography.

FIG. 4 is a graphic representation of H9C2 embryonic cardiocyte cell death when treated with free doxorubicin (Dox) or Dox-DPG at 30 and 10 μg/ml for 72 hrs.

FIG. 5 is a graphic representation of BT-20 human mammary tumor cell death when treated with 10 μg/ml free Dox, 10 μg/ml Dox-DPG, or with 10 μg/ml 2C5-6C31H3 BiSpAb followed by 10 μg/ml Dox-DPG.

FIG. 6 is a graphic representation of the binding of HRP-conjugated rabbit anti-murine IgG antibody (RAMIgG-HRP) to 2G42D7 anti-myosin murine hybridoma cells incubated with 10 μg/ml Bom-BiSpCx, 10 μg/ml 6C31H3 antibody, 10 μg/ml BSA, or culture medium.

FIG. 7 is a graphic representation of the binding of RAMIgG-HRP to H9C2 rat embryonic cardiocytes incubated with 10 μg/ml Bom-BiSpCx, 10 μg/ml 6C31H3 antibody, 10 μg/ml BSA, or culture medium.

FIG. 8 is a graphic representation of the binding of RAMIgG-HRP to PC3 cells incubated with 10 μg/ml Bom-BiSpCx, 10 μg/ml 6C31H3 antibody, or 10 μg/ml BSA.

FIG. 9 is a graphic representation of the binding of DSR-PL to PC3 cells incubated with 10 μg/ml Bom-BiSpCx, 10 μg/ml 6C31H3 antibody, 10 μg/ml BSA, or to untreated cells.

FIG. 10 is a graphic representation of the analysis of Bom-BiSpCx by ELISA using an anti-Bombesin antibody and DTPA-HRP.

FIG. 11 is a graphic representation of the quantification of Bombesin concentration in Bom-BiSpCx by ELISA.

FIG. 12 is a graphic representation of the ratios of tumor versus non-tumor targeting of Tc-DSPL in mice injected with MCA-205 murine fibrosarcoma cells and either pre-treated with Bom-BiSpCx (grey) or not pre-treated (black).

FIG. 13 is a graphic representation of the percentage of total PC-3 cells killed upon pretreatment with Bom-BiSpCx followed by incubation with Dox-DPG; or treated with free doxorubicin alone, Bombesin alone, nanopolymer alone, doxorubicin-loaded nanopolymer alone, or untreated.

FIG. 14 is a graphic representation of the IC₅₀ of Bom-BiSpCx/Dox-DPG (black) and of free doxorubicin (grey) measured in H9C2 embryonic cardiocytes.

### DETAILED DESCRIPTION

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The methods described herein relate to the use of bispecific binding complexes to direct nanopolymers to, e.g., particular cells in vitro and/or in vivo. The bispecific binding complexes specifically bind to a target cell, e.g., to a specific antigen on a target cell, and also bind to the nanopolymer, e.g., to an antigen on the polymer, which is coupled to detection and/or therapeutic agents.

### Definitions

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean a value − or +20% of a given numerical value. Thus, “about 60%” means a value of between 60− (20% of 60) and 60+ (20% of 60) (i.e., between 48 and 70).

The terms “peptide”, “polypeptide” and “protein” are used interchangeably herein.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

The term “treatment” or “treating”, as used herein, refers to administering a therapy in an amount, manner, and/or mode effective to improve a condition, symptom, or parameter associated with a disorder or condition or to prevent or reduce progression of a disorder or condition, either to a statistically significant degree or to a degree detectable to one skilled in the art. An effective amount, manner, or mode can vary depending on the subject and may be tailored to the subject.

The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda. In one embodiment, the antibody is glycosylated.

As used herein, the terms “coupled”, “linked”, “fused”, and “fusion” are used interchangeably. These terms refer to the joining together of two more elements or components by whatever means, including chemical conjugation or recombinant means.

### General

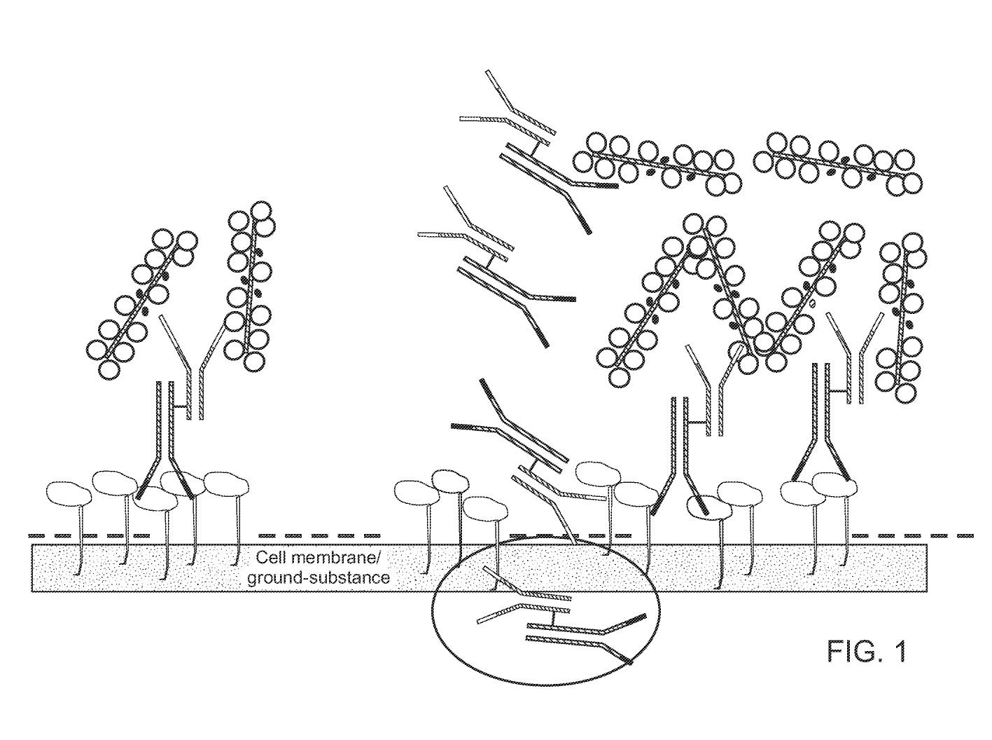

The methods described herein relate to the use of bispecific binding complexes to direct nanopolymers to, e.g., particular cells in vitro and/or in vivo. The bispecific binding complexes specifically bind to a target cell, e.g., to a specific antigen on a target cell, and also bind to the nanopolymer, e.g., to an antigen on the polymer, which is coupled to detection and/or therapeutic agents. An exemplary method is illustrated schematically in FIG. 1.

In some instances, the methods described herein can be used to image cells, e.g., using nanopolymers coupled to a radioactive detection agent. Such nanopolymers target cells with high efficiency and specificity and are cleared quickly, resulting in reduced background activity level. Thus, the methods described herein can be used, e.g., to detect very small lesions by in vivo imaging. In other instances, the methods described herein can be used for therapeutic applications, e.g., when the nanopolymer is conjugated to a therapeutic agent.

### Antibodies

In some of the methods described herein, a bispecific binding complex comprises an antibody-antibody complex, or a bispecific antibody complex. Such bispecific antibody complexes can include a first antibody coupled to a second antibody, with the first antibody specifically binding to a target cell and the second antibody specifically binding to a nanopolymer described herein. In other methods, a bispecific binding complex includes an antibody coupled to a ligand, and the antibody can specifically bind a nanopolymer described herein. The antibodies used in the methods described herein are not limited to any particular antibody, and such antibodies can be obtained commercially or can be produced as described below.

Many types of antibodies, or antigen-binding fragments thereof, are useful in the methods described herein. These antibodies can be of various isotypes, including IgG (e.g., IgG1, IgG2, IgG3, IgG4), IgM, IgA1, IgA2, IgD, or IgE. The antibodies can be full-length (e.g., an IgG1 or IgG4 antibody) or can include an antigen-binding fragment thereof.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody include, e.g., (i) a Fab fragment (a monovalent fragment consisting of the VL, VH, CL and CH1 domains); (ii) a F(ab′)₂ fragment (a bivalent fragment including two Fab fragments linked by a disulfide bridge at the hinge region); (iii) a Fd fragment (consisting of the VH and CH1 domains); (iv) a Fv fragment (consisting of the VL and VH domains of a single arm of an antibody), (v) a dAb fragment (which consists of a VH domain; see, e.g., Ward et al., Nature 341:544-546 (1989)); and (vi) an isolated complementarity determining region (CDR) that retains functionality. Furthermore, although the two domains of the Fv fragment, VL and VH, are coded for by separate genes, they can be joined using recombinant methods by a synthetic linker that enables them to be made as a single protein chain in which the VL and VH regions pair to form monovalent molecules known as single chain Fv (scFv) (see, e.g., Bird et al., Science 242:423-426 (1988); and Huston et al., Proc. Natl. Acad. Sci. USA 85:5879-5883 (1988)).

The antibody can be, e.g., a polyclonal antibody; a monoclonal antibody or antigen binding fragment thereof; a modified antibody such as a chimeric antibody, reshaped antibody, humanized antibody, or fragment thereof. Methods of making polyclonal and monoclonal antibodies are described, e.g., in Harlow et al., Using Antibodies: A Laboratory Manual: Portable Protocol I. Cold Spring Harbor Laboratory (Dec. 1, 1998). For example, an animal can be immunized with a tumor cell described herein to generate antibodies that specifically bind to the tumor cell. Methods for making modified antibodies and antibody fragments (e.g., chimeric antibodies, reshaped antibodies, humanized antibodies, or fragments thereof) are known in the art and can be found, e.g., in Zola, Monoclonal Antibodies: Preparation and Use of Monoclonal Antibodies and Engineered Antibody Derivatives, Springer Verlag (Dec. 15, 2000; 1st Ed.).

In particular instances, the antibody specifically binds to an antigen on a target cell, e.g., a tumor antigen on a tumor cell described herein. Nonlimiting examples of tumor antigens include HER-2 receptor, EGF receptor, VEGF receptor, gastrin releasing peptide receptor, CEA, AFP, tyrosinase, CA-125, Melan-A/MART-1, NY-CO-38, and NY-ESO-1. Other tumor associated antigens are described in, e.g., Stuass et al., Tumor Antigens Recognized by T Cells and Antibodies, Taylor & Francis (London, 2003); Srinivasan et al., Rev. Recent Clin. Trials 1:283-292 (2006); Simpson et al., Nat. Rev. Cancer 5:615-625 (2005); and U.S. Publ. No. 20060194730.

A number of human monoclonal antibodies against tumor associated antigens, including cell surface, cytoplasmic, and nuclear antigens, have been produced and characterized, and any of these can be used in the methods described herein (see, e.g., Yoshikawa et al. (1989) Jpn. J. Cancer Res. (Gann) 80:546-553; Yamaguchi et al. (1987) Proc. Natl. Acad. Sci. USA 84:2416-2420; Haspel et al. (1985) Cancer Res. 45:3951-3961; Cote et al. (1986) Proc. Natl. Acad. Sci. USA 83:2959-2963; Glassy (1987) Cancer Res. 47:5181-5188; Borup-Christensen et al. (1987) Cancer Detect. Prevent. Suppl. 1:207-215; Haspel et al. (1985) Cancer Res. 45:3951-3961; Kan-Mitchell et al. (1989) Cancer Res. 49:4536-4541; Yoshikawa et al. (1986) Jpn. J. Cancer Res. 77:1122-1133; and McKnight et al. (1990) Human Antibod. Hybridomas 1:125-129). Other human monoclonal antibodies are described in Olsson (1985) J. Nat. Cancer Inst. 75:397-404; Larrick and Bourla (1986) J. Biol. Resp. Mod. 5:379-393; McCabe et al. (1988) Cancer Res. 48:4348-4353; Research News (1993) Science 262:841; Ditzel et al. (1994) Cancer 73:858-863; Alonso (1991) Am. J. Clin. Oncol. 4:463-471; and Mack et al. (1995) Proc. Natl. Acad. Sci. USA 92:7021-7025. One exemplary antibody useful in the methods described herein is the pan cancer antibody 2C5 (see, e.g., Iakoubov et al., Oncol. Res. 9:439-446 (1997)).

### Ligands

In some instances, a bispecific binding complex provided by the disclosure includes an antibody coupled to a ligand, where the antibody can specifically bind a nanopolymer described herein. The ligand can specifically bind a cognate binding partner on the target cell, which together form a binding pair. Binding pairs include any combination of molecules that form a complex, including polypeptide/polypeptide and small molecule/polypeptide binding pairs. Non-limiting examples of polypeptide/polypeptide or small molecule/polypeptide binding pairs include a hormone, a cytokine, a polypeptide, a drug, or other antigen, and a cognate receptor or host antibody. Drug/drug receptor binding pairs can be, for example, cocaine/dopamine receptor. Polypeptide/polypeptide receptor binding pairs can be, for example, bombesin/bombesin receptor, acetylcholine/muscarinic receptor, or dopamine/dopamine receptor. Hormone/hormone receptor binding pairs can be, for example, insulin/insulin receptor. Cytokine/cytokine receptor binding pairs can be, for example, tumor necrosis factor (TNF)/TNF Type I or Type 2 receptor, or interleukin 2 (IL-2)/IL-2 receptor.

In particular situations, the ligand binds to a cognate binding partner that is expressed on a target cell, e.g., a target tumor cell. For example, bombesin receptors are over-expressed on the surface of prostate cancer cells as well as other malignant cells (see, e.g., Maina et al., Cancer Imaging 6:153-157 (2006)). Thus, the ligand bombesin can be used to target a nanopolymer described herein to such cells. Other ligands can bind to receptors (e.g., receptors expressed or having increased expression) on tumor cells, such as HER-2 receptor, EGF receptor, VEGF receptor, and gastrin releasing peptide receptor.

### Antibody-Antibody and Antibody-Ligand Coupling

A bispecific binding complex can be generated by coupling a first antibody to a second antibody or coupling an antibody to a ligand. For example, an antibody or antibody portion can be functionally linked (e.g., by chemical coupling, genetic fusion, noncovalent association or otherwise) to one or more other molecular entities, such as another antibody or a ligand as described herein. Nonlimiting examples of crosslinkers that can be used for chemical coupling include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from, e.g., Pierce Chemical Company, Rockford, Ill.

In general, equimolar concentrations of one binding partner will be linked to the other binding partner via covalent bonds as described herein. However, multimeric bispecific complexes may also be generated to improve the avidity of the bispecific complexes, which will provide better targeting molecules.

Methods of coupling are known in the art and can result in, e.g., disulfide bonds, thioether bonds, peptide bonds, or ester bonds between the two antibodies or between the antibody and the ligand. Specific methods are described in, e.g., U.S. Pat. No. 6,451,980; Segal et al., Unit 2.13 in Current Protocols in Immunology, John Wiley & Sons, Inc (2003); Sen et al., J. Hem. Stem Cell Res. 10:247-260 (2001); and Bernatowicz et al., Anal. Biochem. 155:95-102 (1986)).

### Nanopolymers

The disclosure provides methods using nanopolymers, which bind to a bispecific binding complex described herein. In some instances, polycation nanopolymers are used in the methods described herein. Such polycation polymers include, without limitation, poly(allylamine), poly(dimethyldiallyammonim chloride) polylysine, poly(ethylenimine), poly(allylamine), and natural polycations such as dextran amine, polyarginine, chitosan, gelatine A, and/or protamine sulfate. In other instances, polyanion polymers are used, including, without limitation, poly(styrenesulfonate), polyglutamic or alginic acids, poly(acrylic acid), poly(aspartic acid), poly(glutaric acid), and natural polyelectrolytes with similar ionized groups such as dextran sulfate, carboxymethyl cellulose, hyaluronic acid, sodium alginate, gelatine B, chondroitin sulfate, and/or heparin. These polymers can be synthesized using known methods, isolated from natural sources, or, in some cases, commercially obtained.

In certain instances, biodegradable and/or biocompatible polymers are used. These include, without limitation, substantially pure carbon lattices (e.g., graphite), dextran, polysaccharides, polypeptides, polynucleotides, acrylate gels, polyanhydride, poly(lactide-co-glycolide), polytetrafluoroethylene, polyhydroxyalkonates, cross-linked alginates, gelatin, collagen, cross-linked collagen, collagen derivatives (such as succinylated collagen or methylated collagen), cross-linked hyaluronic acid, chitosan, chitosan derivatives (such as methylpyrrolidone-chitosan), cellulose and cellulose derivatives (such as cellulose acetate or carboxymethyl cellulose), dextran derivatives (such carboxymethyl dextran), starch and derivatives of starch (such as hydroxyethyl starch), other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homoploymers, polyoxanones and polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin, silicone rubbers, or polyurethanes, and the like. Other biodegradable materials that can be used include naturally derived polymers, such as acacia, gelatin, dextrans, albumins, alginates/starch, and the like; or synthetic polymers, whether hydrophilic or hydrophobic.

Other nanopolymers include dendrimers, liposomes, long circulating liposomes, micelles, nano-molecules, nano-particles, macromolecules, vesicles, and any molecule that can be modified with drugs for diagnosis or therapy, and others. These vesicles and particles can be synthesized using known methods, isolated from natural sources, or, in some cases, are commercially available.

### Nanopolymer Antigens

In the methods described herein, an antibody in a bispecific binding complex can specifically bind to a nanopolymer described herein. In certain instances, the antibody specifically binds to the nanopolymer directly (e.g., where the polymer includes one or more haptens). In other situations, the nanopolymer is coupled to an antigen, e.g., a small molecule, that is specifically bound by the antibody in the bispecific binding complex.

The methods described herein are not limited by the particular antigen coupled to the nanopolymer, provided that the antibody can specifically bind to such antigen. Nonlimiting examples of antigens include diethylene triaminepentaacetic acid (DTPA), ethylene diamine tetraacetic acid (EDTA), dinitrophenol, and 1,4,7,10-tetraazacyclododecane-N,N′,N″,N′″-tetraacetic acid (DOTA). Other examples of antigens can be small drug molecules, such as doxorubicin, aspirin, tamoxifin, paclitexal, which can function as haptens on carriers to generate specific anti-hapten antibodies. The antigen can be coupled to the nanopolymer using methods described herein, e.g., by chemical coupling.

### Detection Agents

In some instances, the nanopolymers used in the methods of the disclosure are derivatized (or labeled) with a detection agent. Nonlimiting examples of detection agents include, without limitation, fluorescent compounds, various enzymes, prosthetic groups, luminescent materials, bioluminescent materials, fluorescent emitting metal atoms, (e.g., europium (Eu)), radioactive isotopes (described below), quantum dots, electron-dense reagents, and haptens. The detection reagent can be detected using various means including, but are not limited to, spectroscopic, photochemical, radiochemical, biochemical, immunochemical, or chemical means.

Nonlimiting exemplary fluorescent detection agents include fluorescein, fluorescein isothiocyanate, rhodamine, 5-dimethylamine-1-napthalenesulfonyl chloride, phycoerythrin, and the like. A detection agent can also be a detectable enzyme, such as alkaline phosphatase, horseradish peroxidase, β-galactosidase, acetylcholinesterase, glucose oxidase and the like. When a nanopolymer is derivatized with a detectable enzyme, it can be detected by adding additional reagents that the enzyme uses to produce a detectable reaction product. For example, when the detection agent is horseradish peroxidase, the addition of hydrogen peroxide and diaminobenzidine leads to a detectable colored reaction product. A nanopolymer can also be derivatized with a prosthetic group (e.g., streptavidin/biotin and avidin/biotin). For example, a nanopolymer can be derivatized with biotin and detected through indirect measurement of avidin or streptavidin binding. Nonlimiting examples of fluorescent compounds tat can be used as detection reagents include umbelliferone, fluorescein, fluorescein isothiocyanate, rhodamine, dichlorotriazinylamine fluorescein, dansyl chloride, and phycoerythrin. Luminescent materials include, e.g., luminol, and bioluminescent materials include, e.g., luciferase, luciferin, and aequorin.

A detection agent can also be a radioactive isotope, such as, but not limited to, α-, β-, or γ-emitters; or β- and γ-emitters. Radioactive isotopes can be used in diagnostic or therapeutic applications. Such radioactive isotopes include, but are not limited to, iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and gallium (⁶⁷Ga).

The nanopolymers can be radiolabeled using techniques known in the art. In some situations, a nanopolymer described herein is contacted with a chelating agent, e.g., 1,4,7,10-tetraazacyclododecane-N,N′,N″,N′″-tetraacetic acid (DOTA), to thereby produce a conjugated nanopolymer. The conjugated nanopolymer is then radiolabeled with a radioisotope, e.g., ¹¹¹In, ⁹⁰Y, ¹⁷⁷Lu, ¹⁸⁶Re, ¹⁸⁷Re, or ^{99m}Tc, to thereby produce a labeled nanopolymer. In other methods, the nanopolymers can be labeled with ¹¹¹In and ⁹⁰Y using weak transchelators such as citrate (see, e.g., Khaw et al., Science 209:295-297 (1980)) or ^{99m}Tc after reduction in reducing agents such as Na Dithionite (see, e.g., Khaw et al., J. Nucl. Med. 23:1011-1019 (1982)) or by SnCl₂ reduction (see, e.g., Khaw et al., J. Nucl. Med. 47:868-876 (2006)). Other methods are described in, e.g., Lindegren et al., Bioconjug. Chem. 13:502-509 (2002); Boyd et al., Mol. Pharm. 3:614-627 (2006); and del Rosario et al., J. Nucl. Med. 34:1147-1151 (1993).

### Therapeutic Agents

In some methods described herein, the nanopolymer used is conjugated to a therapeutic agent. For example, the therapeutic agent can be a therapeutically active radioisotope described above. Nonlimiting examples of other therapeutic agents include antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU), lomustine (CCNU), cyclothosphamide, busulfan, dibromomannitol, streptozotocin, mitomycin, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Other therapeutic agents include, e.g., cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, calicheamicin, and analogs or homologs thereof.

In particular instances, the therapeutic agent is non-toxic, or exhibits reduced toxicity, when conjugated to the nanopolymer. While not wishing to be bound by theory, it is believed that upon binding of a therapeutic agent-conjugated nanopolymer to a bispecific binding complex (which is itself specifically bound to a target cell), the therapeutic agent-conjugated nanopolymer is internalized by the cell. Upon entry to the cell, the therapeutic agent is released from the nanopolymer and regains its toxicity. Thus, using the methods described herein, cells can be targeted with increased safety.

### Diseases/Disorders

The methods described herein can inhibit the growth, progression, and/or metastasis of hyperproliferative, hyperplastic, metaplastic, dysplastic, and pre-neoplastic diseases or disorders.

By “hyperproliferative disease or disorder” is meant a neoplastic cell growth or proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and cancer. Additional nonlimiting examples of hyperproliferative diseases, disorders, and/or conditions include neoplasms, whether benign or malignant, located in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the term “tumor” or “tumor tissue” refers to an abnormal mass of tissue that results from excessive cell division. A tumor or tumor tissue comprises “tumor cells”, which are neoplastic cells with abnormal growth properties and no useful bodily function. Tumors, tumor tissue, and tumor cells may be benign or malignant. A tumor or tumor tissue can also comprise “tumor-associated non-tumor cells”, such as vascular cells that form blood vessels to supply the tumor or tumor tissue. Non-tumor cells can be induced to replicate and develop by tumor cells, for example, induced to undergo angiogenesis within or surrounding a tumor or tumor tissue.

As used herein, the term “malignancy” refers to a non-benign tumor or a cancer. As used herein, the term “cancer” means a type of hyperproliferative disease that includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to, Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Fibrosarcoma, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, and Wilm's Tumor.

The methods described herein can also be used to treat premalignant conditions and to prevent progression to a neoplastic or malignant state including, but not limited to, those disorders described above. Such uses are indicated in conditions known or suspected of preceding progression to neoplasia or cancer, in particular where non-neoplastic cell growth consisting of hyperplasia, metaplasia, or dysplasia has occurred (see, e.g., Robbins and Angell, Basic Pathology, 2d Ed., W.B. Saunders Co., Philadelphia, pp. 68-79 (1976)).

The methods described herein can further be used to treat hyperplastic disorders. Hyperplasia is a form of controlled cell proliferation, involving an increase in cell number in a tissue or organ, without significant alteration in structure or function. Hyperplastic disorders include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

The methods described herein can also be used to treat metaplastic disorders. Metaplasia is a form of controlled cell growth in which one type of adult or fully differentiated cell substitutes for another type of adult cell. Metaplastic disorders include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

The methods described herein can also be used to treat dysplastic disorders. Dysplasia can be a forerunner of cancer and is found mainly in the epithelia. Dysplasia is a disorderly form of non-neoplastic cell growth, involving a loss in individual cell uniformity and in the architectural orientation of cells. Dysplastic cells can have abnormally large, deeply stained nuclei, and exhibit pleomorphism. Dysplasia can occur, e.g., in areas of chronic irritation or inflammation. Dysplastic disorders include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of the jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders that can be treated by the methods described herein include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

### Pharmaceutical Compositions and Administration

The bispecific binding complexes and nanopolymers described herein can be incorporated into pharmaceutical compositions to be used in the methods described herein. Such compositions can include a bispecific binding complex or a nanopolymer and a pharmaceutically acceptable carrier.

As used herein, a “pharmaceutically acceptable carrier” means a carrier that can be administered to a subject together with a bispecific binding complex or nanopolymer described herein, which does not destroy the pharmacological activity thereof. Pharmaceutically acceptable carriers include, e.g., solvents, dispersion media, coatings, antibacterial and antifungal agents, isotonic and absorption delaying agents, and the like, compatible with pharmaceutical administration. Supplementary active compounds can also be incorporated into the compositions.

Non-limiting examples of pharmaceutically acceptable carriers that can be used include poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid (PLGA), polycaprolactone, polyvalerolactone, poly(anhydrides), copolymers of polycaprolactone with polyethylene glycol, copolymers of polylactic acid with polyethylene glycol, polyethylene glycol; and combinations and blends thereof.

Other carriers include, e.g., an aqueous gelatin, an aqueous protein, a polymeric carrier, a cross-linking agent, or a combination thereof. In another instances, the carrier is a matrix. In yet another instances, the carrier includes water, a pharmaceutically acceptable buffer salt, a pharmaceutically acceptable buffer solution, a pharmaceutically acceptable antioxidant, ascorbic acid, one or more low molecular weight pharmaceutically acceptable polypeptides, a peptide comprising about 2 to about 10 amino acid residues, one or more pharmaceutically acceptable proteins, one or more pharmaceutically acceptable amino acids, an essential-to-human amino acid, one or more pharmaceutically acceptable carbohydrates, one or more pharmaceutically acceptable carbohydrate-derived materials, a non-reducing sugar, glucose, sucrose, sorbitol, trehalose, mannitol, maltodextrin, dextrins, cyclodextrin, a pharmaceutically acceptable chelating agent, EDTA, DTPA, a chelating agent for a divalent metal ion, a chelating agent for a trivalent metal ion, glutathione, pharmaceutically acceptable nonspecific serum albumin, and/or combinations thereof.

A pharmaceutical composition containing a bispecific binding complex or nanopolymer can be formulated to be compatible with its intended route of administration as known by those of ordinary skill in the art. Nonlimiting examples of routes of administration include parenteral, intravenous, intradermal, subcutaneous, oral (e.g., inhalation), transdermal (topical), transmucosal, vaginal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be desirable to include isotonic agents, for example, sugars, polyalcohols such as mannitol, sorbitol, or sodium chloride in the composition. Prolonged absorption of the injectable compositions can be accomplished by including in the composition an agent that delays absorption, for example, aluminum monostearate and gelatin (see, e.g., Remington: The Science and Practice of Pharmacy, 21st edition, Lippincott Williams & Wilkins, Gennaro, ed. (2006)).

Sterile injectable solutions can be prepared by incorporating a bispecific binding complex or nanopolymer in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation include, without limitation, vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, a bispecific binding complex or nanopolymer can be incorporated with excipients and used in the form of tablets, pills, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, a bispecific binding complex or nanopolymer can be delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, but are not limited to, for example, for transmucosal administration, detergents, bile salts, and fusidic acid derivatives. Transmucosal administration can be accomplished through the use of nasal sprays or suppositories. For transdermal administration, the active compounds are formulated into, e.g., ointments, salves, gels, or creams as generally known in the art.

The pharmaceutical compositions containing a bispecific binding complex or nanopolymer can also be prepared in the form of suppositories (e.g., with conventional suppository bases such as cocoa butter and other glycerides) or retention enemas for rectal delivery.

Some pharmaceutical compositions can be prepared with a carrier that protects the bispecific binding complex or nanopolymer against rapid elimination from the body, such as a controlled release formulation, including implants and microencapsulated delivery systems (as described, e.g., in Tan et al., Pharm. Res. 24:2297-2308, 2007). Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially (e.g., from Alza Corp., Mountain View, Calif.). Liposomal suspensions (including liposomes with the bispecific binding complex or nanopolymer on their surface) can also be used as pharmaceutically acceptable carriers. These can be prepared according to methods known to those skilled in the art, e.g., as described in U.S. Pat. No. 4,522,811.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies generally within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. Information for preparing and testing such compositions are known in the art (see, e.g., Remington's The Science and Practice of Pharmacy, 21st edition, Lippincott Williams & Wilkins, Gennaro, ed. (2006)).

In some instances, a therapeutically effective amount or dosage of a bispecific binding complex or nanopolymer can range from about 0.001 mg/kg body weight to about 100 mg/kg body weight, e.g., from about 0.01 mg/kg body weight to about 50 mg/kg body weight, from about 0.025 mg/kg body weight to about 25 mg/kg body weight, from about 0.1 mg/kg body weight to about 20 mg/kg body weight, from about 0.25 mg/kg body weight to about 20 mg/kg body weight, from about 0.5 mg/kg body weight to about 20 mg/kg body weight, from about 0.5 mg/kg body weight to about 10 mg/kg body weight, from about 1 mg/kg body weight to about 10 mg/kg body weight, or about 5 mg/kg body weight.

In other instances, a therapeutically effective amount or dosage of a bispecific binding complex or nanopolymer can range from about 0.001 mg to about 50 mg total, e.g., from about 0.01 mg to about 40 mg total, from about 0.025 mg to about 30 mg total, from about 0.05 mg to about 20 mg total, from about 0.1 mg to about 10 mg total, or from about 1 mg to about 10 mg total.

A physician will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a bispecific binding complex and nanopolymer can include a single treatment or a series of treatments. In one example, a subject is treated with a bispecific binding complex and nanopolymer in the range of between about 0.06 mg to 120 mg, one time per week for between about 1 to 10 weeks, alternatively between 2 to 8 weeks, between about 3 to 7 weeks, or for about 4, 5, or 6 weeks. It will also be appreciated that the effective dosage of a bispecific binding complex and nanopolymer used for treatment may increase or decrease over the course of a particular treatment.

In particular instances, a bispecific binding complex is administered first, followed by administration of a nanopolymer described herein. For example, a bispecific binding complex can be administered first and the nanopolymer is subsequently administered 4 hrs later, 8 hrs later, 12 hrs later, 16 hrs later, 20 hrs later, 24 hrs later, 36 hrs later, 48 hrs later, 72 hrs later, or 4 days, 5 days, 6 days, 7 days, or more days, later.

The pharmaceutical compositions can be included in a container, pack, or dispenser together with instructions for administration.

A person of ordinary skill in the art will appreciate that the pharmaceutical compositions described herein can be formulated as single-dose vials. For example, single-dose vials can be produced containing about 25 μg, about 40 μg, about 60 μg, about 100 μg, about 150 μg, about 200 μg, about 300 μg, or about 500 μg of a bispecific binding complex or nanopolymer-containing pharmaceutical composition described herein. In a further example, single-dose vials can be produced containing a concentration of about 0.5 mM or about 1.0 mM of a pharmaceutical composition described herein.

Treatment of a subject with a therapeutically effective amount of a bispecific binding complex or nanopolymer-containing pharmaceutical composition described herein can be a single treatment, continuous treatment, or a series of treatments divided into multiple doses. The treatment can include a single administration, continuous administration, or periodic administration over one or more years. Chronic, long-term administration can be indicated in many cases. In some instances, a subject is treated for up to one year. In other instances, a subject is treated for up to 6 months. In yet another situation, a subject is treated for up to 100 days. In one example, a subject is treated with a bispecific binding complex and nanopolymer in a time frame of one time per week for between about 1 week to 10 weeks, alternatively between 2 weeks to 8 weeks, between about 3 weeks to 7 weeks, or for about 4 weeks, 5 weeks, or 6 weeks. In other instances, a subject can be treated substantially continuously. In other situations, a subject can be treated once per day, twice per day, once per week, or once per month.

Generally, each formulation is administered in an amount sufficient to suppress or reduce or eliminate a deleterious effect or a symptom of a disorder or condition described herein.

In addition to treating pre-existing disorders, the methods described herein can prevent or slow the onset of such disorders. For example, the bispecific binding complex and nanopolymer described herein can be administered for prophylactic applications, e.g., can be administered to a subject susceptible to or otherwise at risk for a disorder. In some instances, a bispecific binding complex and nanopolymer can be administered to a subject who has a pre-existing disorder and is susceptible to or otherwise at risk for a further disorder.

Suppression of a disorder can be evaluated by any known methods of measuring whether the disorder or a symptom of the disorder is slowed or diminished. Such methods include, e.g., direct observation and indirect evaluation, e.g., by evaluating subjective symptoms or objective physiological indicators.

In some instances, a bispecific binding complex and nanopolymer described herein are administered in combination with one or more additional therapies, e.g., therapeutic agents useful in the treatment of disorders or conditions described herein. For example, the second therapy can include radiation therapy or chemotherapy.

The invention is further illustrated by the following examples. The examples are provided for illustrative purposes only. They are not to be construed as limiting the scope or content of the invention in any way.

### EXAMPLES

### Example 1

### Ultrasensitive In Vivo Imaging of Very Small Cancerous Lesions Using an Antibody-Antibody Complex

A mouse lung metastatic melanoma model was used to detect very small tumor lesions using an antibody-antibody complex and a polymer coupled to an detection agent.

A. Preparation of Bispecific Antibodies Via Thioether Linkage (BiSpAb)

To target cancer cells, the 2C5 pan antibody was used, which recognizes nucleosomes (described in, e.g., Iakoubov et al., Oncol. Res. 9:439-446 (1997)). Whole 2C5 pan cancer antibody (about 2 mg/ml) was treated with 24× molar excess of n-hydroxy succinimide ester of bromoacetic acid (Sigma) for 60 minutes to generate bromoacetylated 2C5 antibody (as described in Bernatowitcz et al., Anal. Biochem. 14:328-336 (1966)). 6C31H3 anti-DTPA antibody (2 mg) was reacted with 100× molar excess of 2-iminothiolane (Trout's reagent) in 25 mM NaBorate, pH 9.1, for 60 minutes at room temperature. The 2C5 and 6C31H3 antibodies were separated from free reagents by Sephadex G-25 column chromatography (10 mL). Equal concentrations of bromoactelyated 2C5 pan-cancer antibody and thiolated 6C21H3 anti-DTPA antibody were mixed and incubated at 4° C. overnight.

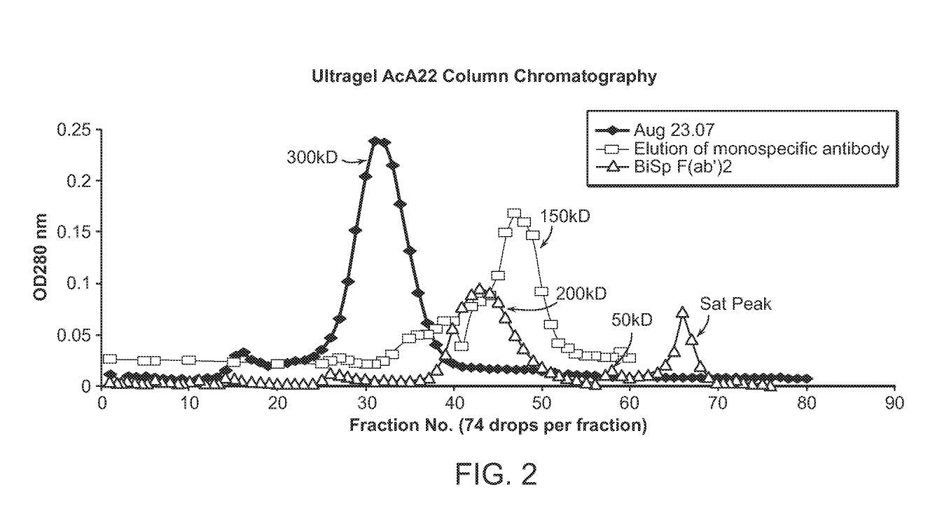

The resulting bispecific anti-pan cancer/anti-DPTA antibodies (“2C5-6C31H3 BiSpAb”) were separated from monospecific antibodies by Ultrogel AcA22 column chromatography (see FIG. 2). The column was precalibrated with monospecific intact IgG antibody, F(ab′)₂, as well as with bispecific intact IgG and F(ab′)₂. The elution profile showed almost 95% formation of 2C5-63C1H3 BiSpAb. There was a small fraction of polymeric bispecific antibodies (peak at fraction #16). The immunoreactivities of the 2C5-63C1H3 BiSpAb prepared by thioether linkage against DTPA and nucleosomes were not different from the immunoreactivities of monospecific 2C5 and 6C31H3 antibodies.

The degree of modification of the antibody with bromoacetic acid was assessed by the TriNitroBenzeneSulfonic acid (TNBS) method, which evaluates the number of lysyl residues modified relative to unmodified lysyl residues on the antibody (described in, e.g., Habeeb, Anal. Biochem. 14:328-336 (1966)). The degree of modification with iminothiolane was assessed by the 5-5′-dithiobis(2-nitrobenzoic acid) (DTNB) method, which determines the optical density of the reaction at 412 nm and multiplies by the extinction coefficient to quantitate the extent of thiolation (described in, e.g., Bush et al., J. Chromatogr. 489:303-311 (1989)).

B. Modification of Polylysine with DTPA

The second main sub-component of the system described in this disclosure is the radiolabeled and negatively charged nanopolymer. Diethylene triamine pentaacetic acid (DTPA)-modified polylysine (PL) was used, which had an approximate size of 15 kDa. This allowed about 73 free amino groups on the polymer to be modified with chelators for chelation to multivalent radioisotopes such as In-111 or Tc-99m. Succinylation of the unmodified epsilon amino groups will render the nanopolymer globally negatively charged. These negatively charged nanopolymers are repelled by the negatively charged cell surfaces and ground substances therefore lowering the non-specific background activity.

Commercially available polylysine was used to prepare DTPA-succinylated polylysine polymers (DSPL) (as described in, e.g., Khaw et al., J. Nucl. Med. 47:868-876 (2006)). Aliquots of 50 μg of polylysine (PL) (14.6 kDa, Sigma Chemical Co.) was solubilized in 0.1 M Na₂CO₃ at pH 8.3, and 100× molar excess of bicyclic anhydride of DTPA in 0.1 ml-0.5 ml of dimethyl sulfoxide (DMSO) was added to the solution. The DTPA-modified PL was subjected to TNBS analysis to determine the number of moles of ε-amino groups of polylysine modified relative to unmodified polylysine (see Habeeb, Anal. Biochem. 14:328-336 (1966)). The reaction mixture was dialyzed against excess (4 L) 0.1M Na₂CO₃ pH 9.6 at 4° C. ON. The DTPA-conjugated PL was then subjected to succinylation with 100× molar excess of succinic anhydride. DTPA-succinyl-PL_{14.6 kDa} was dialyzed in 0.1M Na₂CO₃ pH 9.6 and stored at 4° C. until used. The concentration of DTPA-succinyl-PL (DSPL) was assessed by the Biuret method using unmodified PL to generate a standard curve.

C. Radiolabeling of DSPL with Tc-99m (Tc-DSPL)

Aliquots of 50 μg to 100 μg of DSPL were labeled with 30-50 mCi (1,110-1,850 MBq) of Tc-99m to generate Tc-DSPL, as described in Khaw et al., J. Nucl. Med. 47:868-876 (2006)). A 50 μg aliquot of DSPL in 0.1 M Na₂CO₃ was reacted with 1,110-1,850 MBq of ^{99m}TcO₄⁻ in 50 μg of SnCl₂ and 100 μl of 0.1 N HCl that was previously flushed with N₂ for 10-30 min. After 30 min of incubation, the ^{99m}Tc-labeled DSPL (Tc-DSPL) was separated from free ^{99m}Tc by Sephadex G-25 (10 ml) column chromatography. To obtain higher specific radioactivity, aliquots of 50 μg or 100 μg of DSPL in 0.1M Na₂CO₃ were reacted with double the radioactivity of ^{99m}TcO₄⁻ (2,220-3,700 MBq) in the same volume of 0.1 N HCl previously flushed with N₂ for about 30 mins. After 30 to 60 min of incubation, Tc-DSPL was separated using a Sephadex G-25 column, as described above.

D. In Vivo Gamma Imaging of Small Lesions in Mouse Lung Metastatic Melanoma Model

Nine C57 Bl/6 mice were injected intravenously with 3×10⁵ Bl6F10 murine melanoma cells. 14 days later, the mice were injected with or without 10 μg of 2C5-6C31H3 BiSpAb. The next day, approximately 300 μCi (11 MBq) of Tc-DSPL polymers were injected intravenously. Imaging was initiated at 15 min, at 2 hrs, and again at 24 hrs. In vivo and ex vivo target (T) to background (B) activity ratios were obtained by computer planimetry.

In 5 mice with sub-optimal Tc-DSPL labeling, blood activity cleared in 2 hrs. Tumor uptake was seen in one mouse with 2C5-6C31H3 BiSpAb/Tc-DSPL. No lung lesions were seen at necropsy in the other twp mice. No in vivo or ex vivo lesions were seen in two control mice. In two 2C5-6C31H3 BiSpAb/Tc-DSPL-treated mice (one died from over-anesthesia) or two Tc-DSPL-treated mice, in vivo and ex vivo tumor activities were seen in the former and not in the latter. Mean in vivo T/B ratio for 2C5-6C31H3 BiSpAb/Tc-DSPL (14.1+/−2.4 [+/−SD]) was significantly greater than that of Tc-DSPL alone (3.7+/−1.9, P<0.02). Ex vivo T/B ratios for 2C5-6C31H3 BiSpAb/Tc-DSPL and DSPL alone were 6.1+/−0.03 and 2.5+/−0.77, respectively (P<0.02). Lesions in the lungs were less than 1.5 mm in diameter.

Thus, signal amplification by Tc-DSPL and 2C5-6C31H3 BiSpAb targeting enabled visualization of very small metastatic melanoma lung lesions by in vivo gamma imaging.

### Example 2

### Enhanced Targeted Drug Delivery of Chemotherapeutic Agents Using an Antibody-Antibody Complex

The 2C5-6C31H3 BiSpAb described in Example 1 was used in combination with a polyglutamic acid nanopolymer covalently linked to doxorubicin to achieve targeted delivery of doxorubicin. These nanopolymer-conjugated drug molecules were tested in embryonic cardiocytes to determine whether cardiotoxicity was reduced with respect to free doxorubicin at the same concentrations. In addition, the tumorotoxicity of these nanopolymer-conjugated drug molecules was tested in BT-20 human mammary tumor cells.

A. Preparation of N-Terminal DTPA-Modified Dox Loaded Polyglutamic Acid (Dox-DPG)

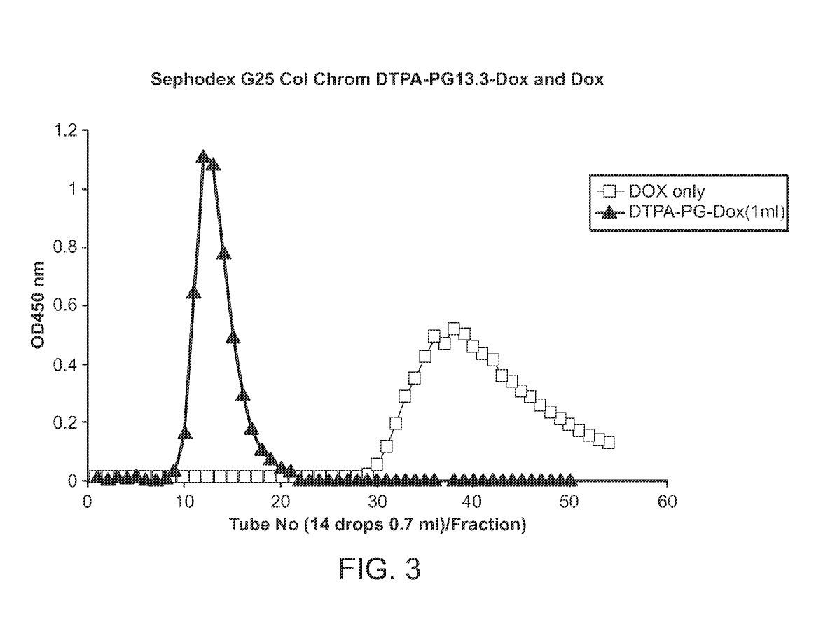

50 mg of (10 mg/ml) polyglutamic acid (PGA, m.w. 13.3 kDa) in 0.1 M NaHCO₃, pH 8.6, was reacted with 3× molar excess of anhydride of DTPA (Sigma). DTPA conjugated PGA (D-PGA) was dialyzed in 0.1 M Phosphate buffered saline (PBS) pH 7.4. DTPA incorporation was demonstrated by ELISA using anti-DTPA antibody and compared to binding of the antibody to DTPA-BSA. Then 4.5-9 mg of Dox was covalently linked via peptide bonds to the carboxylic acids of 10 mg of D-PGA using 9-18 mg water soluble 1-ethyl-3-(3-dimethylaminopropyl)carbodiimide-HCL (EDC). The resulting Dox-DPG nanopolymers were separated from free Doxorubicin by Sephadex G-25 column chromatography. After incubation at room temperature overnight, the reaction mixture showed almost 100% incorporation of Doxorubicin on the nanopolymers, as assessed by Spehadex G-25 column chromatography (13 cm×0.5 cm; see FIG. 3). Free Doxorubicin was eluted in the salt volume. 4.5 mg and 9 mg of Doxorubicin (8.287×10⁻⁶ moles or 2×8.287×10⁻⁶ moles) were incorporated in 10 mg of 13.3 kD polyglutamic acid (7×10⁻⁷ moles). This resulted in approximately 11.8 moles or 23.6 moles of Doxorubicin on 1 mole of polyglutamic acid. Dox concentration on D-PGA was assessed by OD₄₉₀ nm and comparison to OD 490 nm of serial dilutions of Dox to generate a standard curve.

B. In Vitro Assay for Cardiotoxicity in H9C2 Embryonic Rat Cardiocytes

H9C2 embryonic rat cardiocytes were cultured in 10% Fetal Clone DMEM (F-DMEM) at 37° C. in 25 ml sterile culture flasks until 90% confluent. Then the cells were dissociated from the flask bottoms by trypsin digestion. The cells were washed and counted in a hemocytometer. Aliquots of 40,000 H9C2 cells were delivered to each well of 9 well culture plates in 1 ml of F-DMEM. After incubating for 24 hrs, the culture media were removed and fresh culture media containing 10 μg/ml or 30 μg/ml of Dox or Dox-DPG were added and incubation at 37° C. continued for another 48 hrs. The cells in the culture media were collected and centrifuged to separate the cells from the media. 100 ml of Tryptan Blue dye solution was then added, the cells were washed, and cell counts were determined using a hemocytometer.

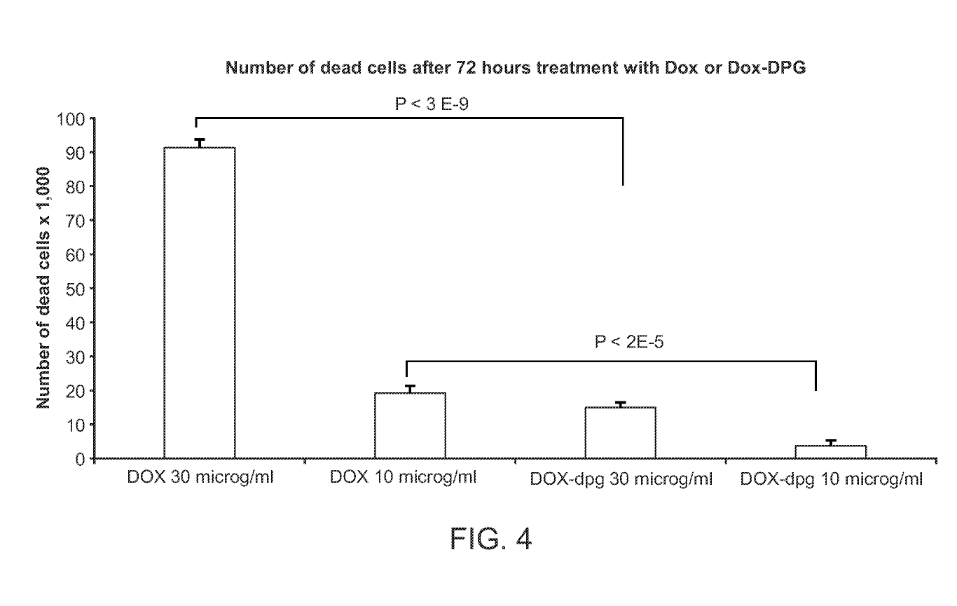

As depicted in FIG. 4, cardiotoxicity of H9C2 cells was reduced by about 7× when incubated with Dox-DPG relative to incubation with free Doxorubicin molecules at the same concentrations.

C. In Vitro Assay for Tumorotoxicity in BT-20 Human Mammary Tumor Cells

BT-20 human mammary tumor cells were grown in 10% Fetal Clone DEME. When the cells reached about 80% confluence, the cells were harvested by trypsin digestion and replated in 9 well culture plates at 400,000 cells per well. After 24 hrs, the culture media was removed and in one set of wells was replaced with media containing 10 μg/ml of 2C5-6C31H3 BiSpAb (prepared as described in Example 1). In two other sets, the media was replaced with media lacking 2C5-6C31H3 BiSpAb. Each set was prepared in triplicate. After 24 hrs of incubation, the medium was removed and washed 3× with medium lacking antibody. Medium containing 10 μg/ml of free Dox was then added to a medium-only set of wells, and media containing 10 μg/ml of Dox-DPG was added to one set with and one set without 2C5-6C31H3 BiSpAb. The cultures were incubated for either 3 hrs, 12 hrs, or 24 hrs, at which times the cells in the media were collected and viability was determined by counting cells with Tryptan Blue exclusion criteria in a hemocytometer.

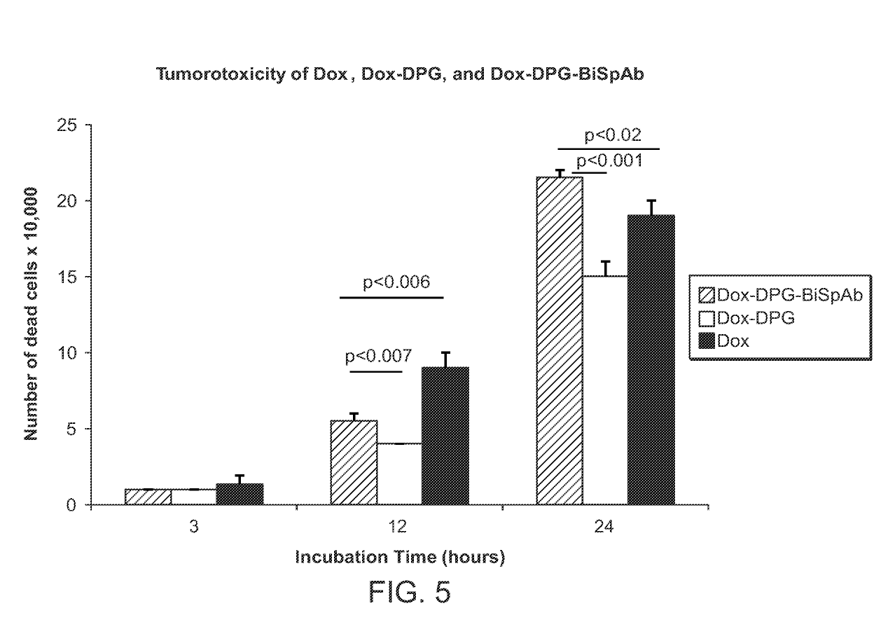

FIG. 5 depicts the tumorocidal activities between free Doxorubicin (Dox), nanopolymer-conjugated Doxorubicin (Dox-DPG), and nanopolymer-conjugated Doxorubicin targeted with the bispecific antibody (Dox-DPG-BiSpAb) at 3 hrs, 12 hrs, and 24 hrs. As shown in FIG. 5, in the early stage of incubation, free Doxorubicin was more effective in killing tumor cells. However, from 12 hrs to 24 hrs, tumor cell death was significantly higher when treated with Dox-DPG-BiSpAb. When this study is translated to an in vivo tumorocidal experiment, free Doxorubicin is not able to maintain the same serum concentration because it is cleared from the blood. Therefore, the killing activity of doxorubicin also decreases over time with decreasing blood concentrations. However, with bispecific antibody targeting and selective binding to the tumor sites, the high specific activity of the Dox-DPG-BiSpAb allows its enhanced internalization into the tumor cells by endocytosis. Endosolysosomal enzymes degrade the polyglutamic acid and release free Doxorubicin into the cytoplasm of the tumor cells. As demonstrated in FIG. 5, this leads to an enhanced targeted killing of the tumor cells with an increase in BT-20 cell death greater than that achieved with free Doxorubicin (p<0.02).

### Example 3

### Ultrasensitive and Selective In Vitro, Ex Vivo, and In Vivo Detection and Imaging of Cancer Cells Using an Antibody-Ligand Complex

A. Methods

1. Preparation of Bombesin-6C31H3 Bispecific Complexes (Bom-BiSpCx)

Intact 6C31H3 anti-DTPA antibody was modified with 100× molar excess iminothiolane. Bombesin was modified with 24× molar excess N-hydroxy-succinimide ester of bromoacetic acid (as described in Varvarigou et al., Can. Biother. Radiopharm. 19:219-229 (2004); and Bernatowicz et al., Anal. Biochem. 155:95-102 (1986)). The addition of 100× moles excess of Bombesin to antibody resulted in 1:1 Bombesin to 6C31H3 bispecific complexes via thioether bonds. The antibody activity and Bombesin concentrations were assessed by ELISA using either DTPA-BSA or anti-Bombesin antibody and compared to standard curves. Specifically, the Bombesin concentration of serial dilutions of Bom-BiSpCx starting at 1 μg/ml was determined by ELISA using Bombesin (1 μg/ml) and 6C31H3 antibody (1 μg/ml) as controls. Bombesin of the Bom-BiSpCx (1 μg/ml) was captured by an anti-Bombesin antibody, resulting in a positive reaction with DTPA-HRP (which is specific for the 6C31H31 antibody of the Bom-BiSpCx).

2. Preparation of DTPA-Succinyl Rhodamine Labeled Polylysine Nanopolymer (DSR-PL)

DTPA-conjugated polylysine was prepared as described in Example 1. The percent of lysyl residue modification was calculated by comparison to unmodified PL. Residual free lysyl residues were modified by the addition of 100× molar excess of rhodamine isothiocynate. The remaining free lysyl residues were succinylated with 100× molar excess of succinic anhydride.

3. In Vitro Cell-Based Binding Assay

The cell lines H9C2 (rat embryonic cardiocytes), 2G42D7 (anti-myosin murine hybridomas), and PC3 (human prostate cancer) were grown in 10% DMEM medium. The cells were incubated with varying concentrations of Bom-BiSpCx at 37° C. for 1 hr. After washing, targeting on cells was demonstrated with horse radish peroxidase (HRP)-conjugated rabbit anti-murine IgG antibody (RAMIgG-HRP), specific to murine anti-DTPA antibody, and K-blue color reagent. Control assays included of pretreatment with 6C31H3 antibody, bovine serum albumin (BSA), or culture medium alone.

Epifluorescent microscopy was performed on PC3 cells incubated with 10 μg/ml Bom-BiSpCx, 10 μg/ml Bombesin, 10 μg/ml 6C31H3, or culture medium for 1 hr at 4° C. The cells were then washed and incubated with DSR-PL at 4° C. for 1 hr.

4. Data Analysis

Data were analyzed with Adobe Photo Shop 7. The fluorescent intensity of each cell was computer planimetered and mean pixel density was determined for a total of cells each. Regions without cells were planimetered and pixel density determined for background. This background pixel density was then subtracted from the mean cellular pixel densities. Statistical significance was assessed by Student's t-test at 95% confidence interval.

5. In Vivo Targeting of Xenograft Tumors with Bom-BiSpCx

SCID mice (20 g) hosting MCF-7 fibrosarcoma tumors (10-12 mice in each group) were injected with 10 μg of Bom-BiSpCx. After waiting for Bom-BiSpcCx to clear from the circulation, Tc-DSPL (24 MBq, prepared as described in Example 1) was administered intravenously, and serial gamma imaging was performed for 3 hrs. After radiotracer injection, images were acquired at 5 min, 15 min, 30 min, 60 min, 120 min, and 180 min. Blood pool clearance and biodistribution and tumor activity were determined by scintillation counting.

In other experiments, 10⁵ MCA-205 murine fibrosarcoma cells were injected subcutaneously into the shoulder region of C57B l/6 mice. After 14 d, mice were injected intravenously with 10 μg of Bom-BiSpCx (n=3) or saline (n=4). The next day, 37 MBq of Tc-DSPL (approximately 1 ng) were injected intravenously while under Ketamine and xylazine anesthesia. Mice were imaged at the time of injection for 500 sec. Then each mouse was imaged again at 10 min and/or 15 min. The animals were returned to their cages and allowed to recover from anesthesia. At 24 hrs, the mice were re-anesthetized and imaged for 1500 sec each. Each image was analyzed using ImageJ program from NIH. The tumor, contralateral, heart, and thigh regions were planimetered and normalized for time of acquisition when necessary. Tumor to contralateral pixel density ratios were plotted against time after intravenous injection of Tc-DSPL. Microsoft Excel was used to fit trend lines and determine Student's t-test.

6. Tissue Processing

Upon completion of gamma imaging, animals were euthanized by an overdose of IP injection of sodium pentobarbital (100 mg/kg) or ketamine/xylazine (100 mg/kg and 10 mg/kg respectively). Tumors were excised, weighed, counted in a gamma scintillation counter (model 1282 Compugamma; LKB Instruments, Inc., Gaithersburg, Md.), and then frozen in histo-prep frozen tissue embedding media for preparation of frozen sections for immunohistochemical and histological examination. Tissue samples (blood, heart, lung, liver, spleen, kidney, stomach, intestines, skeletal muscle and bone) were obtained, weighed, and counted in the gamma scintillation counter. Aliquots of the radiolabeled polymer were saved to determine the injected dose. 1:100 dilution of this aliquot was made and 10 μl and 100 μl samples were counted with the tissue samples. 100 μl of the radiolabeled polymer containing 2 μg of polymer with specific radioactivity of 170 MBq/μg was used to inject experimental animals. Thus, 1 μl of the injected sample contained 3.4 MBq. This was added to 999 μl of PBS to obtain a solution of 3.4 MBq/ml (10 μl aliquots of this dilution contained 0.034 MBq). Aliquots of the standards were included as duplicate or triplicate samples before and after, as well as in the middle region of the tissue samples counted. The average of the standards were used to determine the dosages in the tissue samples. Since both the tissues and the standard samples were counted at about the same time, the comparison of tissue counting data to the standards was automatically corrected for physical decay. The counts per minutes of the standard samples were multiplied by 10,000 to obtain the injected dose. To determine the % injected dose/g, the tissue count-activity per gram was divided by the total injected dose then multiplied by 100. One mCi or 37 MBq is theoretically 2.2×10⁹ dpm (or about 60,000 dpm per Bq). Therefore, a dose of 340 MBq is 2.04×10⁸ dpm. An aliquot equivalent to 1/10,000 dilution as described above having 2.04×10⁴ dpm was counted immediately in the gamma counter, and the actual cpm obtained was divided by 2.04×10⁴ dpm to determine the efficiency of the gamma counter. The efficiency of the gamma counter was used to transform cpm into dpm.

B. Results

Cells incubated with Bom-BiSpCx showed a positive reaction with RAMIgG-HRP significantly greater than when incubated with free 6C31H3 antibody, BSA, or culture medium alone. A comparison was undertaken to determine the affinity of Bombesin for different cell lines containing G protein coupled receptors (GPCRs) on their surface. Rabbit anti-murine antibody conjugated with HRP (RAMIgG-HRP), which binds to the murine 6C31H3 antibody, was used to demonstrate specific binding. Only the Bombesin-6C31H3 antibody complexes left bound to the surface receptors after washing allows the RAMIgG-HRP to bind and give positive signals, whereas free 6C31H3 antibody or albumin does not bind to the cells and will be washed away.

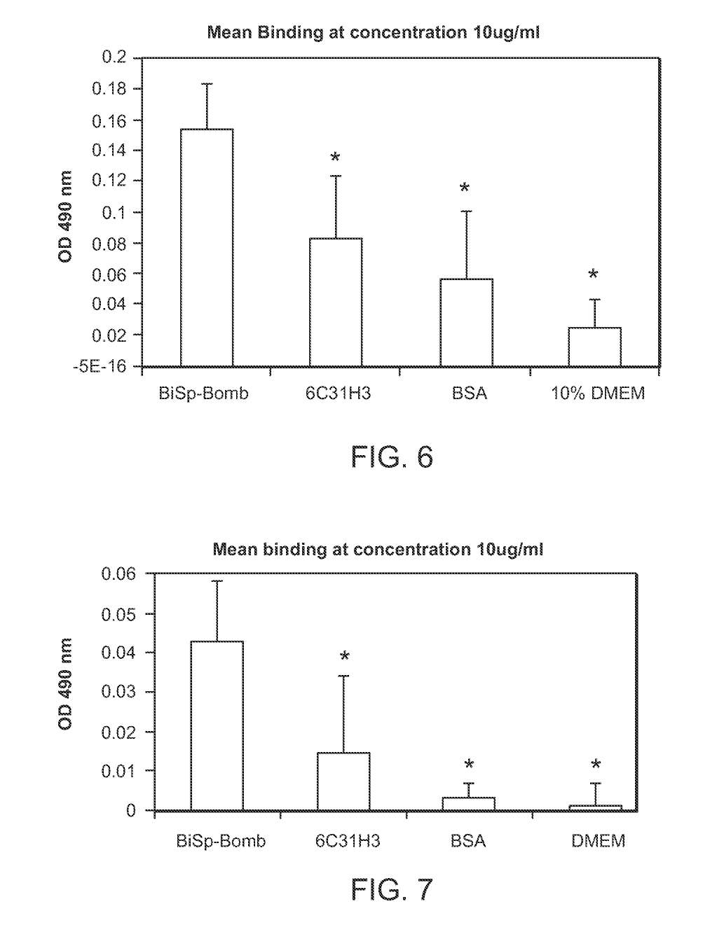

Hybridoma 2G42D7 cells, H9C2 rat embryonic cardiocytes, and PC3 cells were incubated with 10 μg/ml Bom-BiSpCx, 10 μg/ml 6C31H3 antibody, 10 μg/ml BSA, or culture medium alone. As demonstrated in FIG. 6, 2G42D7 cells targeted with Bom-BiSpCx showed a positive reaction with RAMIgG-HRP (O.D. 490 nm=0.154+/−0.0286), significantly greater than free 6C31H3 antibody (0.083+/−0.0399, p<0.02), BSA (0.0451+/−0.1114, p<0.03), or culture medium alone (0.025+/−0.0174, p<0.002) (* indicates p<0.05 compared to Bom-BiSpCx). As shown in FIG. 7, H9C2 rat embryonic cardiocytes targeted with Bom-BiSpCx also showed positive reaction with RAMIgG-HRP (O.D. 490 nm=0.04275+/−0.0154), which was significantly greater than free 6C31H3 antibody (0.0145+/−0.019, p<0.02), BSA (0.003+/−0.003, p<0.0004), or culture medium alone (0.00075+/−0.0059, p<0.002) (* indicates p<0.05 compared to Bom-BiSpCx). These results indicate that 2G42D7 and H9C2 cells have GPCRs on their surface.

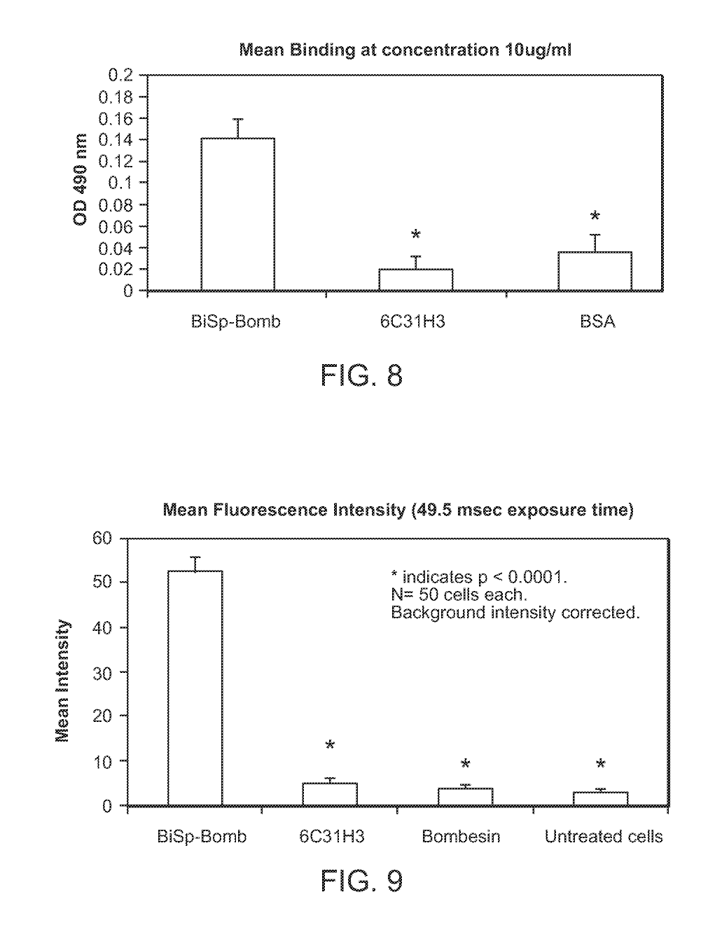

As depicted in FIG. 8, PC3 cells targeted with Bom-BiSpCx also showed positive reaction with RAMIgG-HRP signal (O.D. 490 nm=0.141+/−0.017), significantly greater than free 6C31H3 antibody (0.0202+/−0.012, p<0.00001) or BSA (0.036+/−0.016, p<0.00001) (* indicates p<0.05 compared to Bomb-BiSpCx). Further, PC3 cells demonstrated higher signal at the same concentration of Bom-BiSpCx, indicating the presence of high levels of Bombesin receptors. This result indicated that the signal observed with Bom-BiSpCx was due to the binding of the Bombesin molecule on the complex with the GPCR cell surface Bombesin receptor.

To demonstrate the binding of DTPA-conjugated polymers to Bom-BiSpCx, epifluorescent microscopy analysis was undertaken. PC3 cells were incubated with 10 μg/ml Bom-BiSpCx, 10 μg/ml 6C31H3 antibody, 10 μg/ml Bombesin, or were untreated, and were then incubated with DSR-PL, as described above. As depicted in FIG. 9, only PC3 cells pre-targeted with Bom-BiSpCx showed significantly greater epifluorescence relative to controls. PC3 cells targeted with Bom-BiSpCx showed an intensity of DSR-PL signal (52.48+/−3.14) significantly greater than free 6C31H3 antibody (4.8638+/−1.30, p<0.0001), free Bombesin (3.652+/−0.974, p<0.0001), or untreated cells (2.452+/−0.596) (* indicates p<0.0001 compared to Bom-BiSpCx).

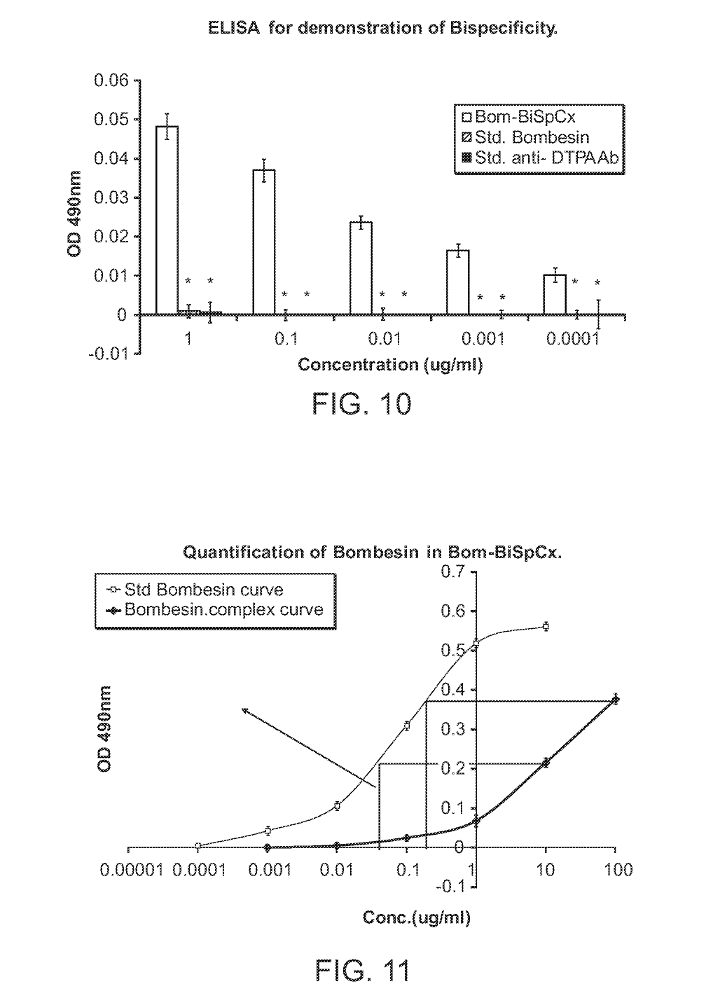

An ELISA assay was used to determine the bispecificity of Bom-BiSpCx, using an anti-Bombesin antibody as the capture antibody and DTPA-HRP. As depicted in FIG. 10, Bombesin of the Bom-BiSpCx (1 μg/ml) was captured by the anti-Bombesin antibody and resulted in a positive reaction with DTPA-HRP (O.D. 490 nm=0.04825±0.00329). This was significantly greater than Bombesin (0.001±0.00172) or DTPA alone (0.0007±0.0025) (* indicates p<0.05 compared to Bom-BiSpCx at respective concentrations). Further, the amount of Bombesin in the Bom-BiSpCx, as quantitated by ELISA, was about 0.5 to 1 mole of Bombesin per mole of antibody (FIG. 11).

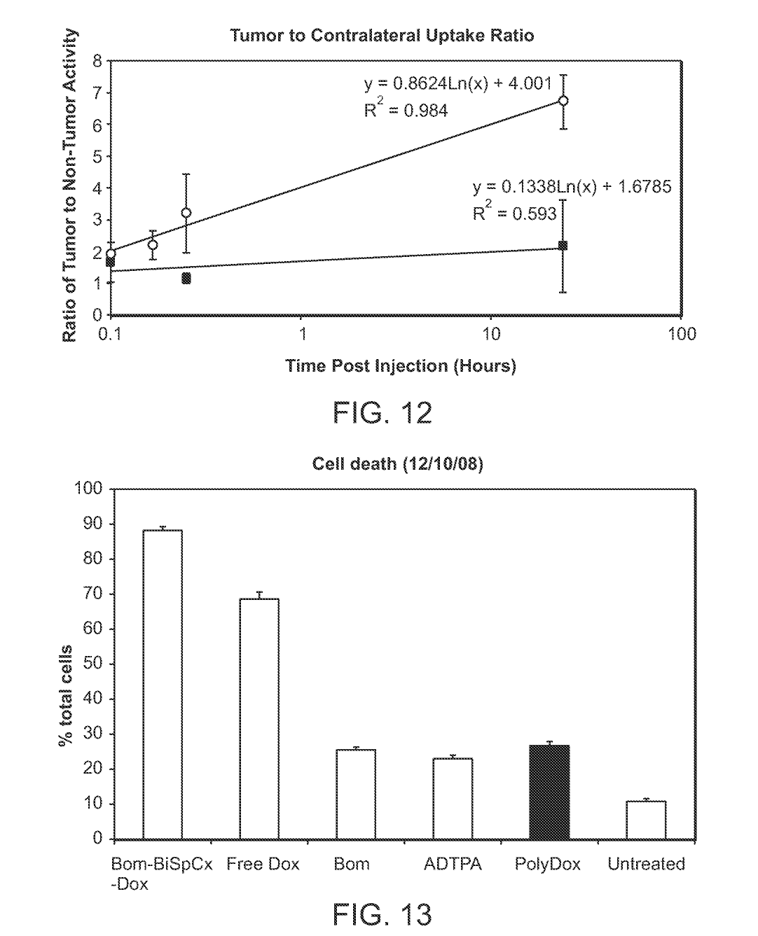

Pretargeting with Bom-BiSpCx enabled the visualization of MCF-7 fibrosarcoma allografts in mice. Visualization of BT-20, PC-3, and murine B16F10 melanoma cells was also achieved by pretargeting with Bom-BiSpCx. In mice injected with MCA-205 murine fibrosarcoma cells, tumors pretargeted with Bom-BiSpCx were visualized as early as 15 min, but the mean uptake ratio at 24 hrs (6.7+/−0.84) was significantly greater than the mean uptake ratio of Tc-DSPL in control mice not pre-treated with Bom-BiSpCx (2.16+/−1.45, p<0.01). An increase in the uptake ratio of Tc-DSPL with time in mice pre-targeted with Bom-BiSpCx (y=0.86 Ln(x)+4.001, R2=0.98) was observed, whereas control mice injected showed a correlation of y=0.13 Ln(x)+1.68 (R2=0.593) (FIG. 12). These results demonstrate that Bom-BiSpCx can be used to pre-target tumor cells with Tc-DSPL.

Together, these findings demonstrate that Bom-BiSpCx detects ligand-receptor interactions.

### Example 4

Enhanced Targeted Drug Delivery of Chemotherapeutic Agents Using an Antibody-Ligand Complex

A. Methods

1. In Vitro Therapeutic Efficacy in PC-3 Cell Cultures of Bom-BiSpCx/Dox-DPG

PC-3 cancer cells were grown in 10% fetal clone-DMEM until they reached about 80% confluence. The cells were then harvested by trypsin digestion and re-plated in 6 well culture plates at 40,000 cells per well. After attaining 70% confluency, the medium was replaced with or without 10 μg of Bom-BiSpCx (prepared as described in Example 3) in triplicates to quadruplicates. After 24 hr of incubation at 37° C. in 5% CO₂, the media was changed to media containing 10 μg/ml of free Dox or Dox-DPG (prepared as described in Example 2). The cells were further incubated at 37° C. for 0-3 hr, 3-12 hr, or 12-24 hr, at which time the cells in each well were collected and the number of live and dead cells determined by Trypan blue exclusion test in a Hemocytometer. Negative control cells were treated with monospecific 2C5 antibody, 6C21H3 antibody, Bombesin, or culture media alone. Positive control cells were treated with 10 μg/ml of free Dox. Each assay was repeated at least 3 times in triplicate or quadruplicate.

2. IC₅₀ of Dox-DPG and Dox in H9C2 Embryonic Cardiocytes

H9C2 embryonic rat cardiocytes cultured in 10% Fetal Clone DMEM (F-DMEM) at 37° C. in 25 ml sterile culture flasks until 90% confluent in triplicate were dissociated by Trypsin digestion. The cells were washed in F-DMEM. Aliquots of 40,000 H9C2 cells in 1 ml F-DMEM were delivered to each well of a 6 well culture plate. After incubation for 24 hr, the culture medium was removed and fresh medium containing 2.5 μg/ml, 5 μg/ml, 10 μg/ml, 15 μg/ml, and 30 μg/ml of Dox-DPG or free Dox were added and incubated at 37° C. for 24 hr. Cell death was assessed by Trypan Blue exclusion test.

B. Results

As shown in FIG. 13, the antitumor activity of Bom-BiSpCx/Dox-DPG, as evaluated in vitro in cultured PC-3 cancer cells, was improved relative to that of free doxorubicin. The Bom-BiSpCx/Dox-DPG resulted in the death of 88.2% of PC-3 cancer cells, compared to 68.6% for free doxorubicin, representing an improvement of 29%.

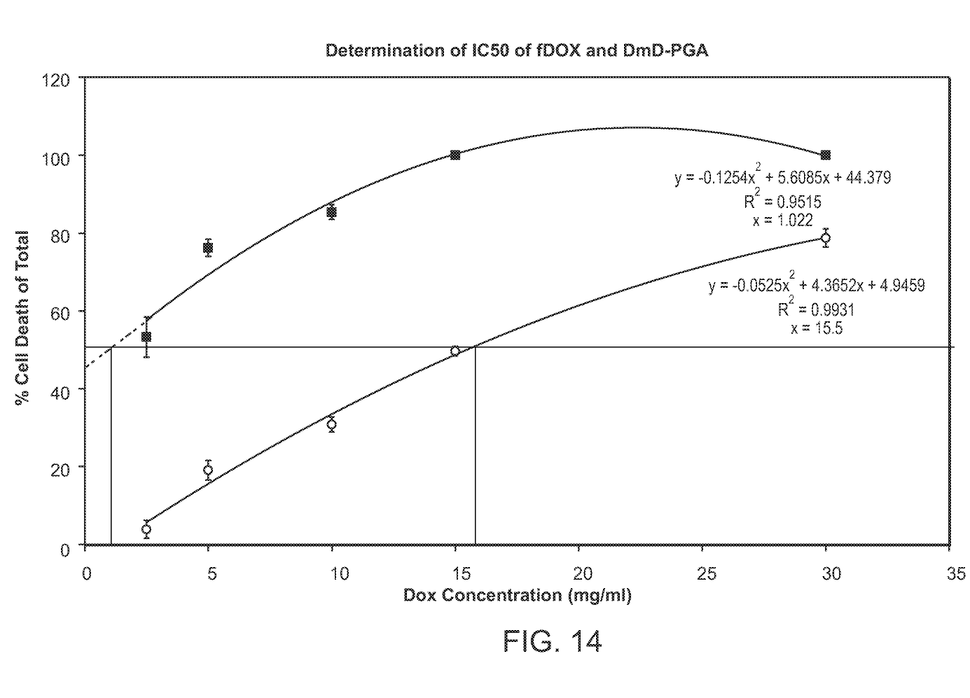

Further, the cardiotoxicity of Bom-BiSpCx/Dox-DPG, as evaluated in vitro in cultured H9C2 cardiocytes, was reduced relative to that of free doxorubicin (see FIG. 14). The IC₅₀ for Bom-BiSpCx/Dox-DPG was 15.5 μg/ml in H9C2 rat embryonic cardiocytes, whereas the IC₅₀ for free doxorubicin was 1.02 μg/ml for free doxorubicin. This represents a 15.2× reduction in cardiotoxicity.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting a cancer cell in a subject, the method comprising:
(a) administering to the subject a first antibody covalently linked to a second antibody;
(b) administering to the subject a polymer comprising an detection agent, the first antibody specifically binding the polymer and the second antibody specifically binding an antigen on the cancer cell; and
(c) detecting the detection agent, thereby detecting the cancer cell.

2. The method of claim 1, wherein the polymer is coupled to an antigen.

3. The method of claim 2, wherein the first antibody specifically binds the antigen coupled to the polymer.

4. The method of claim 3, wherein the polymer is polylysine.

5. The method of claim 3, wherein the antigen coupled to the polymer is diethylene triaminepentaacetic acid (DTPA).

6. The method of claim 1, wherein the cancer cell antigen is a pan cancer antigen.

7. A method of detecting a cell, the method comprising:
(a) contacting the cell with an antibody covalently linked to a ligand;
(b) contacting the cell with a polymer comprising an detection agent, the antibody specifically binding the polymer, and the ligand specifically binding a receptor on the cell; and
(c) detecting the detection agent, thereby detecting the cell.

8. The method of claim 7, wherein the ligand is bombesin.

9. The method of claim 7, wherein contacting steps (a) and (b) comprise administering the antibody and the polymer to a subject.

10. The method of claim 7, wherein the cell is detected in vitro.

11. A method of treating a cell, the method comprising:
(a) contacting the cell with an antibody covalently to a ligand, the ligand binding a receptor on the cell; and
(b) contacting the ligand-bound cell with a polymer coupled to the therapeutic agent, the antibody specifically binding the polymer,
the therapeutic agent thereby being delivered to, and treating, the cell.

12. The method of claim 11, wherein the ligand is bombesin.

13. The method of claim 11, wherein the cell is in a subject, and the antibody-ligand conjugate and the polymer-therapeutic agent complex are administered to the subject.

14. The method of claim 11, wherein the cell is treated in vitro.

15. The method of claim 11, wherein the therapeutic agent is doxorubicin.

16. The method of claim 11, wherein the polymer further comprises a detection agent and the method further comprises detecting the detection agent, thereby detecting the cell.
